(19) United States
(12) Patent Application Publication
(10) Pub. No.: US 2019/0175336 A1
(43) Pub. Date: Jun. 13, 2019

(54) METHOD AND APPARATUS FOR PRACTICE OF TAVR EMPLOYING AN EXPANDABLE MESH-LIKE CATHETER

(71) Applicant: Bedrosian Global, LLC, Reno, NV (US)

(72) Inventors: David Rizik, Scottsdale, AZ (US); Bert S. Bedrosian, Malibu, CA (US)

(21) Appl. No.: 16/176,591

(22) Filed: Oct. 31, 2018

Related U.S. Application Data
(63) Continuation of application No. 15/202,122, filed on Jul. 5, 2016, now abandoned.
(60) Provisional application No. 62/254,547, filed on Nov. 12, 2015.
(60) Provisional application No. 62/190,319, filed on Jul. 9, 2015.

(51) Int. Cl. A61F 2/24

(52) U.S. Cl. CPC A61F 2/243; A61F 2/2433; A61F 2250/0059

(57) ABSTRACT

The present invention is a method of practicing transcatheter valve replacement (TAVR) employing an expandable hollow mesh-like catheter which may be inserted through an aorta to the proximity of a diseased or degenerated aortic valve in a retracted form and then expanded to compress the valve to open it up. Subsequently a new valve will be placed on the collapsed mesh, will be fed through the aorta, and the mesh will be expanded to push the leaflets of the old valve aside. The mesh does not block blood flow, eliminating the need to instigate a rapid heartbeat as required in the prior art procedure employing a balloon rather than the mesh.

### CROSS-REFERENCE TO RELATED APPLICATIONS

This application is a continuation of 15/202,122 filed on Jul. 5, 2016 which claims priority of U.S. Provisional Patent Application No. 62/190,319 filed Jul. 9, 2015, and U.S. Provisional Patent Application No. 62/254,547 filed Nov. 12, 2015; the contents of each of which are incorporated herein by reference.

### BACKGROUND OF THE INVENTION

Aortic stenosis (AS) describes a condition in which the heart's aortic valve is narrowed. The aortic valve is the portal or doorway through which blood exits the heart from its pumping chamber, the left ventricle. This narrowing of the aortic valve prevents the valve from opening fully, which obstructs blood flow from the heart into the main blood vessel of the body (the aorta) and onward to the rest of the body.

Surgical aortic valve replacement has been the time honored therapy for the treatment of AS. For those patients deemed to represent a high risk for standard surgical aortic valve replacement (to correct the problem), a non-surgical, catheter based alternative has been developed called transcatheter aortic valve replacement (TAVR). TAVR involves the placement of a valve prosthesis over a catheter, generally inserted through a blood vessel in the leg to replace the diseased native valve.

At present, balloon aortic valvuloplasty (BAV) is a standard procedure for dilating the diseased or degenerated aortic valve prior to implantation of TAVR device. The purpose of BAV is to prepare the stenotic native valve, partially opening it, in order to make TAVR a safer, more efficacious procedure (easier procedure with fewer complications). The BAV device, generally a balloon dilatation catheter, is engineered and designed to work in concert with TAVR.

There are limits to and potential complications associated with balloon technology used in the performance of BAV prior to TAVR including but not limited to obstruction of flow through the aorta, embolic debris which can be dislodged from the aortic valve leading to stroke and other complications as well as the possibility of obstruction of flow to the coronary arteries during inflation. BAV also requires implantation or insertion of a temporary pacemaker in order to stabilize the balloon within the aortic valve complex during inflation of the balloon.

Therefore an alternative to the use of an inflatable balloon in BAV would potentially have multiple advantages to the current technology.

In the practice of TAVR, following the dilation of the degenerated aortic valve by BAV, and the removal of the deflated balloon catheter, a new aortic valve will be placed on a delivery system employing a deflated balloon and will be pushed into the old aortic valve. The balloon will then be inflated while the heart is stabilized by speeding up the heartbeat using the temporary pacemaker and the new valve will push the leaflets of the degenerated valve aside.

This valve insertion process is subject to the same complications as the BAV process.

### SUMMARY OF THE INVENTION

The present invention, a mesh like, non-balloon based, dilating catheter provides a much safer and efficient apparatus for deploying a TAVR valve by replacing the standard balloon apparatus. It is a solid, mesh-like or scaffold-like material that would serve as a “pretreatment” of the native aortic valve, compressing the area prior to implantation of the transcatheter heart valve (THV) and during the subsequent implantation it can be expanded or deployed without the necessity of the requirement of a temporary pacemaker.

This mesh-like, flow support device would be hollow and therefore blood flow would neither be restricted from entering the aorta nor the coronary arteries unlike the current balloon technology used for the purposes of BAV. The instigation of a rapid heartbeat with a temporary pacemaker would not be required in order to stabilize the balloon within the aortic valve complex, the area to be compressed, since a balloon is no longer involved in the equation. This configuration provides a much safer and efficient device compared to the current model. The design is also intended to limit the possibility of debris from breaking off the native disease valve, reducing the potential for stroke and other embolic complications associated with BAV and TAVR.

### BRIEF DESCRIPTION OF THE DRAWINGS

FIG. 1 is a perspective view of the distal end of a mesh-like catheter of the present invention in collapsed form; and

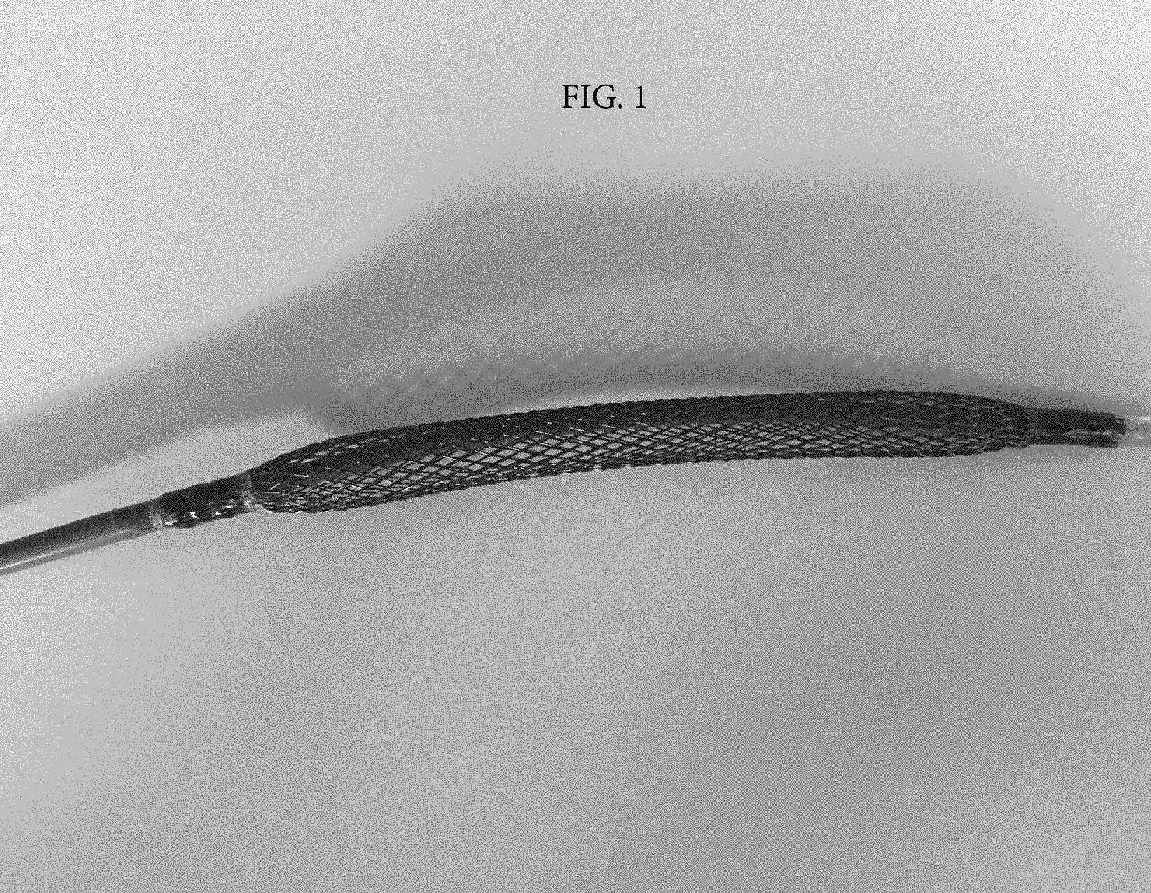

FIG. 2 is a perspective view of the distal end of a catheter of the present invention in expanded form.

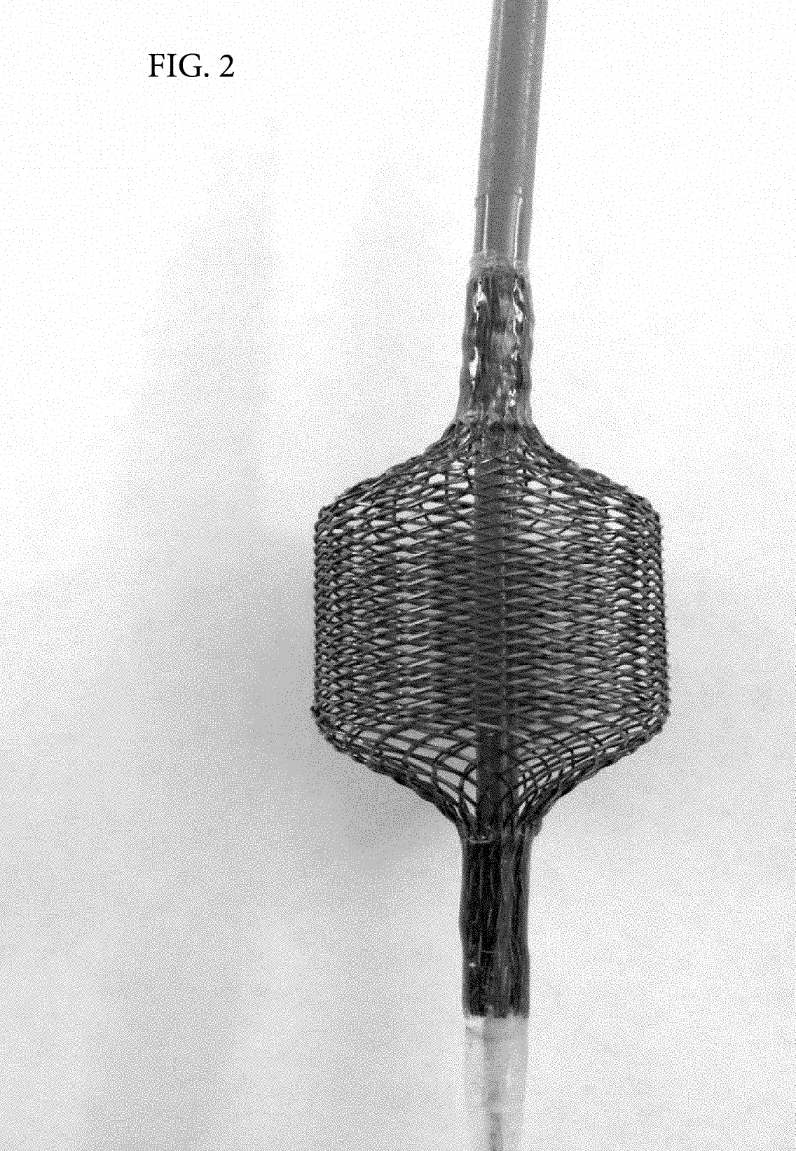

### DETAILED DESCRIPTION OF A PREFERRED EMBODIMENT OF THE INVENTION

The expandable mesh device is fed into proximity to the diseased aortic valve using a transfemoral, transapical or a transaortic technique. In the transfemoral technique, a sheath is inserted into the femoral artery in the patient's leg or slightly higher. The folded expandable mesh device is then inserted into the sheath and fed up the artery to the diseased valve. A wire attached to the valve is then pulled from the lower end of the sheath to pull the ends of the mesh toward one another and expand the sheath so that it compresses the valve to open it up. In the transapical technique the mesh device is fed through an incision between the ribs; and in the transaortic technique through an incision in the chest.

The method and apparatus of the present invention can also be employed in the following situations:
1. Mechanical valvuloplasty of the Mitral valve for Mitral stenosis, whether as a stand alone therapy or as a pretreatment for transcatheter valve replacement;
2. Mechanical valvuloplasty of the Tricuspid valve for tricuspid stenosis, whether as a stand alone therapy or as a pretreatment for transcatheter valve replacement;
3. Mechanical valvuloplasty of the Pulmonic valve for Pulmonic stenosis, whether as a stand alone therapy or as a pretreatment for transcatheter valve replacement;
4. Treatment of peripheral vascular disease, whether in the aorta or the extremities (in the setting of arterial stenosis).

The same expandable mesh based devices used during BAV, or a similar device with a different design or dimensions, may be used during valve implantation.

The expandable mesh device may be of the type disclosed in U.S. Pat. No. 5,449,372 to Schmaltz et al., which is intended for use as a temporary stent, but incorporates the features of the expandable mesh device employed with the method of the present invention.

The method of this invention is applicable to any of the various forms of the heart entry for which TAVR is employed such as transfemoral, transapical, or transaortic.

1. The method of surgical replacement of a diseased or degenerated aortic valve, comprising:
inserting a catheter supporting a folded mesh, expandable, hollow structure into the aortic valve and passing it to proximity of the valve to be replaced;
expanding the mesh hollow structure against the valve to be replaced without restricting blood flow to the heart, to compress and dilate the valve to be replaced;
withdrawing the catheter from the aorta; and
inserting a replacement valve into the heart through the aorta.
